(19) United States
(12) Patent Application Publication
(10) Pub. No.: US 2024/0325795 A1
(43) Pub. Date: Oct. 3, 2024

(54) HEAD WEARABLE AIR PURIFIER

(71) Applicant: Dyson Technology Limited, Wiltshire (GB)

(72) Inventor: Nikian Naji AGHABABAIE, London (GB)

(21) Appl. No.: 18/293,979

(22) PCT Filed: Jul. 27, 2022
(86) PCT No.: PCT/GB2022/051971

§ 371 Date: Jan. 31, 2024

(30) Foreign Application Priority Data
Aug. 3, 2021 (GB) 2111186.9

(51) Int. Cl. A62B 18/00; A61L 2/10; A61L 2/24; A61L 2/26; A62B 18/08; A62B 23/02

(52) U.S. Cl. CPC A62B 18/003; A61L 2/10; A61L 2/24; A61L 2/26; A62B 18/006; A62B 18/08; A62B 18/084; A62B 23/02; A61L 2202/11; A61L 2202/14

(57) ABSTRACT

The disclosure describes a head wearable air purifier including a filter assembly, a motor-driven impeller for creating an airflow through the filter assembly to obtain a filtered airflow downstream of the filter assembly, and an ear assembly arranged to be worn over an ear of a user, the ear assembly comprising at least one light source for emitting light in a far UVC portion of the electromagnetic spectrum. The air purifier also includes a nozzle attached to the ear assembly, the nozzle comprising a nozzle inlet arranged to receive the filtered airflow downstream of the filter assembly, and a nozzle outlet for emitting the received filtered airflow from the head wearable air purifier. The at least one light source is arranged to illuminate at least part of the nozzle for the decontamination thereof.

### FIELD OF THE INVENTION

The present invention relates to a head wearable air purifier. In particular, the invention relates to the use of light in a far UVC portion of the electromagnetic spectrum to decontaminate at least part of a nozzle of the head wearable air purifier.

### BACKGROUND

Air pollution is an increasing problem and a variety of air pollutants have known or suspected harmful effects on human health. The adverse effects that can be caused by air pollution depend upon the pollutant type and concentration, and the length of time that a person is exposed to the polluted air. For instance, relatively high air pollution levels can cause immediate health problems such as aggravated cardiovascular and respiratory illness, whereas long-term exposure to polluted air can have permanent health effects such as loss of lung capacity and decreased lung function, and the development of diseases such as asthma, bronchitis, emphysema, and possibly cancer. Airborne pathogens are also an issue that can cause various health risks when individuals are exposed to (i.e. breathe in) such pathogens.

In locations with particularly high levels of air pollution, many individuals have recognised the benefits of minimising their exposure to these pollutants and have taken to wearing face masks with the aim of filtering out at least a portion of the pollutants present in the air before it reaches the mouth and nose. These face masks range from basic dust masks that merely filter out relatively large dust particles, to more complex air-purifying respirators that the air pass through a filter element or cartridge. However, as these face masks typically cover at least a user's mouth and nose they can make normal breathing more laborious and can also make it difficult for the user to speak effectively to others, such that there is some reluctance to make use of such face masks on a day-to-day basis despite the potential benefits.

As a consequence, there have been attempts to develop air purifiers that can be worn by the user but that do not require the user's mouth and nose to be covered. For instance, one design for a head wearable air purifier includes two ear assemblies to be worn over respective ears of a user and connected by a headband, similar to a typical pair of headphones. Each ear assembly may include a motor-driven impeller and a filter assembly, where the motor-driven impeller creates an airflow through the filter assembly to obtain a filtered airflow downstream thereof. The filtered airflow may then be directed to a user's mouth. For instance, in one design the head wearable air purifier includes a nozzle or visor that is connected at each end to the respective ear assemblies and, in use, is positioned next to the user's mouth and nose. The nozzle may receive the filtered air downstream of the filter assembly and direct the air to an opening or nozzle output that is positioned adjacent to the user's mouth and nose in use so as to deliver filtered air to the user.

An issue with such an air purifier is that air drawn into the filter assembly may be obtained from an environment that may contain many different kinds of contamination. Larger dirt and dust particles may be filtered out, but smaller contaminants such as bacteria and other smaller microbes are drawn in too. Such microbial contamination may then gather and grow in and around the nozzle. Also, continuous breathing of a user onto the nozzle over a time period in which the air purifier is being used can increase the risk of bacterial growth on the nozzle. Cleaning the hard surfaces of an air purifier may be performed using a wet cloth, for instance. However, this can actually lead to an increase in levels of microbial contamination. In any case, it may be difficult, or not possible, to access certain parts of a nozzle to be decontaminated with such a cloth, for instance an internal part of the nozzle. A further constraint when considering how the nozzle of such air purifiers may be cleaned is that there is generally a relatively small amount of space in the nozzle in which physical devices to aid decontamination may be fitted or positioned.

It is against this background to which the present invention is set.

### SUMMARY OF THE INVENTION

According to an aspect of the invention there is provided a head wearable air purifier, comprising a filter assembly and a motor-driven impeller for creating an airflow through the filter assembly to obtain a filtered airflow downstream of the filter assembly. The head wearable air purifier comprises an ear assembly arranged to be worn over an ear of a user, the ear assembly comprising at least one light source for emitting light in a far UVC portion of the electromagnetic spectrum. The head wearable air purifier comprises a nozzle attached to the ear assembly, the nozzle comprising a nozzle inlet arranged to receive the filtered airflow downstream of the filter assembly, and a nozzle outlet for emitting the received filtered airflow from the head wearable air purifier. The at least one light source is arranged to illuminate at least part of the nozzle for the decontamination thereof.

The head wearable air purifier may comprise a light guide arranged to guide light emitted from the at least one light source to illuminate the at least part of the nozzle to be decontaminated.

The light guide may comprise at least one light pipe to guide light emitted from the at least one light source through the at least one light pipe.

At least part of the light guide may be disposed in the ear assembly.

The light guide may be arranged to guide the light emitted from the at least one light source to a connection surface of the ear assembly. The connection surface of the ear assembly may be arranged to face a corresponding connection surface of the nozzle.

The light guide may be narrower at an end adjacent to the at least one light source than at an opposite end adjacent to the connection surface of the ear assembly. In this way, the light guide may be arranged to spread the emitted light from the at least one light source to span a majority of a length of the connection surface of the ear assembly.

The connection surface of the ear assembly may be formed from a material that allows the emitted light to pass therethrough.

The connection surface of the ear assembly may be formed from a plastic material. Optionally, the connection surface may be formed from acrylic, polycarbonate, polypropylene or may include the use of polylactic acid.

The ear assembly may comprise an attachment portion that includes the connection surface and is for attaching the ear assembly to the nozzle. The attachment portion may be arranged to receive the filtered airflow downstream of the filter assembly and to provide the received filtered airflow to the nozzle. The at least one light source may be disposed adjacent to the attachment portion.

The at least one light source may be attached to the attachment portion.

At least part of the light guide may be disposed in the nozzle.

The light guide may comprise one or more shaped features, for reflecting the emitted light, on an internal surface of the head wearable air purifier.

The nozzle may be arranged to receive light from the at least one light source in the ear assembly into an internal part of the nozzle.

The nozzle may comprise an attachment portion that includes the nozzle inlet and is for attaching the nozzle to the ear assembly, and a visor portion including the nozzle outlet. The light from the at least one light source in the ear assembly may be received into a space defined between the attachment and visor portions of the nozzle.

Light from the at least one light source in the ear assembly may irradiate an internal surface of the nozzle.

The head wearable air purifier may comprise electrical switching means arranged to switch between a closed state, in which the at least one light source is activated to emit light, and an open state, in which the at least one light source is deactivated and does not emit light.

The electrical switching means may be arranged to automatically switch from the open state to the closed state upon the nozzle being attached to the ear assembly.

In the closed state a motor of the air purifier may be powered to drive the impeller to create the airflow through the filter assembly, and in the open state the motor may not drive the impeller.

The nozzle may be rotatable relative to the ear assembly when the nozzle is attached to the ear assembly. The electrical switching means may be arranged to automatically switch from the open state to the closed state upon the nozzle being moved from a first position to a second position, different from the first position, relative to the ear assembly.

The nozzle may be generally arcuate in shape.

The filter assembly and the motor-driven impeller may be part of the ear assembly. Optionally, the filter assembly and the motor-driven impeller may be generally conical or frustoconical in shape.

The at least one light source may be in the form of one or more LEDs.

The at least one light source may be configured for emitting light with a wavelength of about 222 nm.

The ear assembly may be a first ear assembly arranged to be worn over a first ear of the user. The head wearable air purifier may comprise a second ear assembly arranged to be worn over a second ear of a user. The second ear assembly may comprise at least one second light source for emitting light in a far UVC portion of the electromagnetic spectrum, the at least one second light source may be arranged to illuminate at least part of the nozzle for the decontamination thereof.

The nozzle output may be arranged, in use, adjacent to a user's mouth and nose.

The head wearable purifier may comprise a headband connecting the first and second ear assemblies.

### BRIEF DESCRIPTION OF THE DRAWINGS

Examples of the invention will now be described with reference to the accompanying drawings, in which:

FIG. 1 shows a schematic view of a head wearable air purifier according to an example of the invention;

FIG. 2 shows a cross section of the head wearable air purifier of FIG. 1.

FIG. 3 shows a cross section of an ear assembly of the head wearable air purifier of FIG. 1;

FIG. 4 shows part of the view of the head wearable air purifier of FIG. 1;

FIG. 5 shows part of the view of the ear assembly of FIG. 3;

FIGS. 6(a) and 6(b) show perspective views of a nozzle attachment portion, a light source and a light guide of the head wearable air purifier of FIG. 1; and,

### DETAILED DESCRIPTION

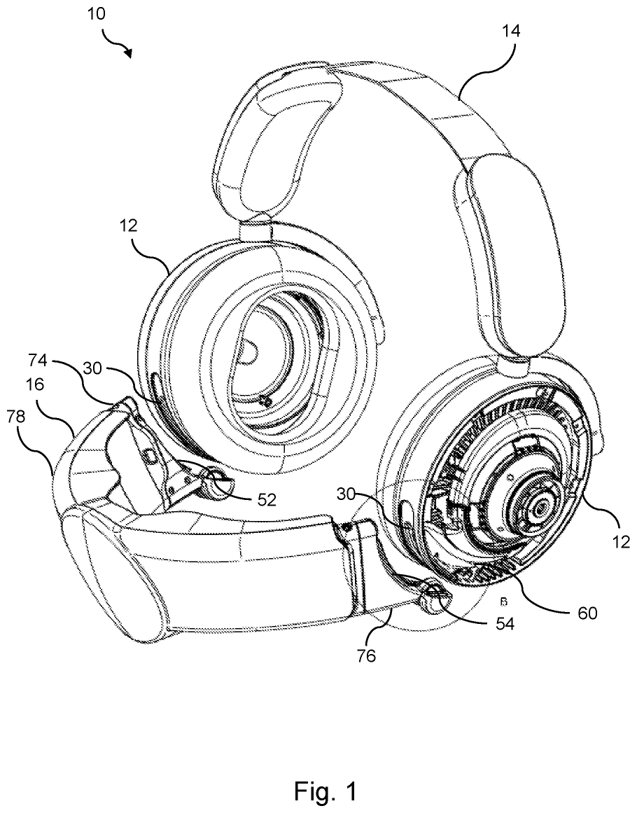

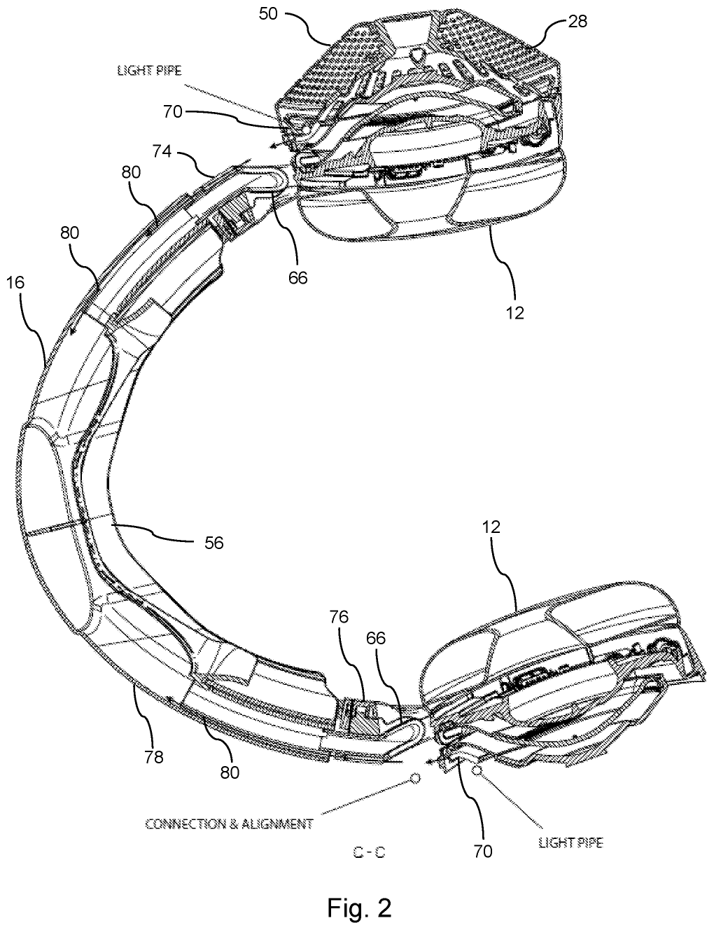

FIGS. 1 and 2 show external and sectional views of an example of a head wearable air purifier 10. The head wearable air purifier 10 includes a pair of generally cylindrical ear assemblies or ear cups 12 connected to an arcuate headband 14, and a nozzle or visor 16 that that extends between, and is connected at, opposite ends to the ear assemblies 12.

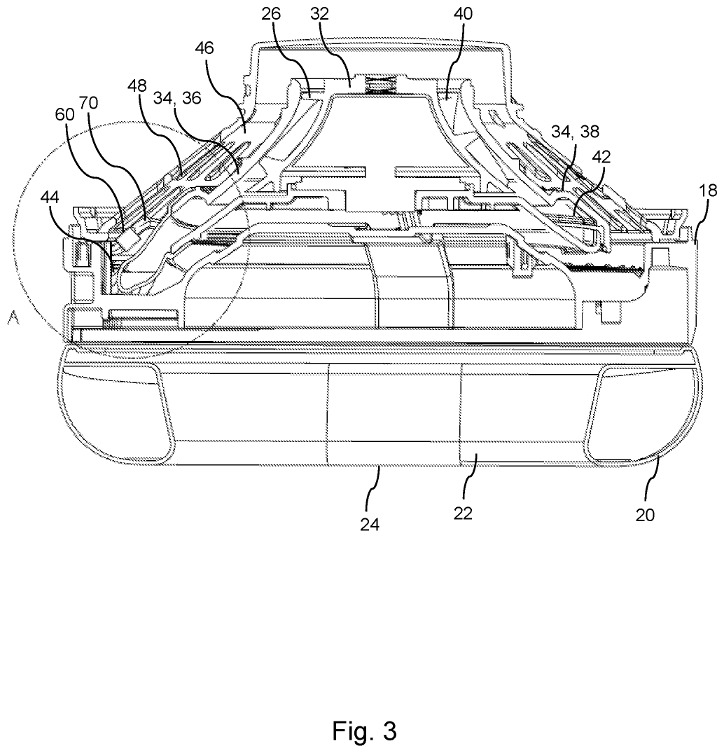

With additional reference to FIG. 3—which shows a sectional view of one of the ear assemblies 12—each ear assembly 12 includes a housing 18 and an ear pad 20 attached to the housing 18, with the housing 18 and ear pad 20 together defining a cavity 22 having an opening 24. An optional speaker or acoustic driver unit may be attached to the housing 18 such that it is at least partially exposed to the cavity 22.

Each of the ear assemblies 12 may include a motor-driven impeller 26 disposed within the housing 18 that is arranged to create an airflow through the housing 18. The housing 18 may therefore be provided with an air inlet 28 (see FIG. 2) through which an airflow can be drawn into the housing 18 by the motor-driven impeller 26, and an air outlet 30 (see FIG. 1) for emitting the airflow from the housing 18.

A filter assembly (not shown in FIG. 3) may be disposed within the housing 18 such that the airflow generated by the motor-driven impeller 26 passes through the filter assembly and such that the airflow emitted from the ear assembly 12 is filtered/purified by the filter assembly. The filter assembly is therefore located downstream (relative to the airflow generated by the motor-driven impeller 26) of the air inlet 28 of the housing 18 and upstream of the air outlet 30. In the described example, the filter assembly is also located upstream of, or relative to, the motor-driven impeller 26.

Each of the ear assemblies 12 may include one or more circuit boards upon which various circuitry is disposed or mounted. For instance, this electronic circuitry may include motor control circuitry that is arranged to control a rotational speed of a motor 32 that drives the impeller 26.

A generally frustoconical impeller casing 34 containing both the impeller 26 and the motor 32 may be disposed in the ear assembly 12. In some examples, the impeller casing 34 may be regarded as being part of the housing 18 of the ear assembly 12. This impeller casing 34 may include a generally frustoconical impeller housing 36 surrounding the impeller 26 and the motor 32, and an annular volute 38 fluidically connected to a base of the impeller housing 36, where the annular volute 38 is arranged to receive air exhausted from the impeller housing 36. The impeller housing 36 is provided with an air inlet 40 through which air can be drawn by the impeller 26, and an air outlet 42 through which the air is emitted from the impeller housing 36 to the annular volute 38. The air inlet 40 of the impeller housing 36 is provided by an aperture/opening at the small diameter end of the impeller housing 36 and the air outlet 42 is provided by an annular slot formed around a large diameter end of the impeller housing 36.

The annular volute 38 may include a spiral (i.e. gradually widening) duct that is arranged to receive the air exhausted from the impeller housing 36 and to guide the air to an air outlet of the volute 38. The air outlet of the volute 36 may be fluidically connected to the air outlet 30 of the ear assembly 12. The term ‘volute’ as used herein refers to a spiral funnel that receives the fluid being pumped by an impeller and increases in area as it approaches a discharge port. The air outlet of the volute 38 therefore provides an efficient and quiet means for collecting the air that is exhausted from the circumferential annular slot that that forms the air outlet 42 of the impeller housing 36.

In the described example, the impeller 26 is a mixed flow impeller that has a generally conical or frustoconical shape. The impeller 26 is hollow such that a rear/back side of the impeller 26 defines a generally frustoconical recess. The motor 32 is then nested/disposed within this recess. The impeller 26 may be a semi-open/semi-closed mixed flow impeller, i.e. having a back shroud only. The back shroud of the impeller 26 then defines the recess within which the motor 32 is nested/disposed.

The impeller casing 34 may optionally be supported/suspended within the housing 18 by a plurality of resilient supports 44 that reduce the transmission of vibrations from the impeller casing 34 to the housing 18. To do so, the plurality of resilient supports 44 may each comprise a resilient material such as an elastomeric or rubber material.

The filter assembly may be mounted so that it is provided upstream of the impeller 26 and is arranged to be nested over the impeller casing 34. A filter seat or frame 46 supports the filter assembly 32, which may include one or more filter elements, e.g. a particulate filter element and/or a chemical filter element.

The filter seat 46 may be provided with a plurality of apertures 48 that allow air to pass from a front surface of the filter seat 46 to a rear/back surface of the filter seat 46, with the front surface being arranged to support the filter assembly over the plurality of apertures 48. The filter seat 46 then further defines an air passageway or channel between the rear/back surface of the filter seat 46 and the air inlet 40 of the impeller casing 34 that is arranged to guide air to the air inlet 40 of the impeller casing 34. This air passageway may be provided by a cavity defined between the rear/back surface of the filter seat 46 and a front surface of the impeller casing 34. Air therefore passes through the filter assembly before it can pass through the apertures 48 in the filter seat 46 and into the air passageway that leads to the air inlet 40 of the impeller casing 34.

In the described example, the filter seat 46 may be located over the impeller housing 36, with the impeller housing 36 partially disposed within a volume defined by a back of the filter seat 46. In particular, the filter seat 46 may include a generally frustoconical peripheral portion and a generally cylindrical central portion. The generally frustoconical peripheral portion of the filter seat 46 is provided with the plurality of apertures 48 and is arranged to support one or more generally frustoconical filter assembly elements over the plurality of apertures 48. The impeller housing 36 may be at least partially disposed within the generally cylindrical central portion of the filter seat 46. In particular, the air inlet 40 of impeller housing 36 may be disposed within a volume defined by a back of the cylindrical central portion of the filter seat 46.

Structural components or parts of the ear assembly 12, such as the housing 18, impeller casing 34 and filter frame 46, may together be regarded as a body of the ear assembly. One or more of the structural components may be formed from a plastics material, e.g. acrylic. In particular, the filter frame 46 may be formed, at least in part, from acrylic.

The (body of the) ear assembly 12 may further include an outer cover 50 (see FIG. 2) that is arranged to fit over the filter assembly. The outer cover 50 may be provided with an array of apertures that allow air to pass through the outer cover, such that the apertures define an air inlet of the outer cover 50 (and therefore the air inlet 28 of the ear assembly 12). These apertures are sized to prevent larger particles from passing through to the filter assembly and blocking, or otherwise damaging, the filter elements. Alternatively, in order to allow air to pass through, the outer cover 50 could comprise one or more grilles or meshes mounted within windows in the outer cover 50. It will also be clear that alternative patterns of arrays are possible.

Returning to FIGS. 1 and 2, in the described example a first open end 52 of the nozzle 16 is connected at the air outlet 30 of a first one of the ear assemblies 12. The nozzle 16 extends away from the first ear assembly 12 and may assume an arcuate shape so that the opposite, second open end 54 of the nozzle 16 connects at the air outlet 30 of a second one of the ear assemblies 12. The nozzle 16 may be arranged such that, when the purifier 10 is worn by a user with the first ear assembly 12 over a first ear of the user and the second ear assembly 12 over a second ear of the user, the nozzle 16 can extend around the user's face, from one side to the other, and in front of (i.e. adjacent to) a mouth of the user. In particular, the nozzle 16 may extend around the jaw of the user, from adjacent to one cheek to adjacent the other cheek, without contacting the mouth, nose or surrounding regions of the user's face. It may therefore be preferable that at least a portion of the nozzle 16 is formed of a transparent or partially transparent material so that the user's mouth is visible through the nozzle 16 so as to avoid limiting the user's ability to speak to others in a clear manner. For instance, a central portion of the nozzle 16 could be made from a flexible, transparent plastic such as a polyurethane, whilst the two end portions could each made from a stiff, transparent plastic such as a polyethylene terephthalate glycol-modified (PETG). Alternatively, the entire nozzle 16 could be formed from a single transparent or partially transparent material.

The nozzle 16 is provided with an air outlet 56 for emitting/delivering the filtered air to a user. For instance, the air outlet 56 of the nozzle 16 can comprise an array of apertures formed in a section of the nozzle 16, with these apertures extending from an interior passage defined by the nozzle 16 to an exterior surface of the nozzle 16. Alternatively, the air outlet 56 of the nozzle 16 may comprise one or more grilles or meshes mounted within windows in the nozzle 16.

In use, the purifier 10 is worn by a user with the first ear assembly 12 over a first ear of the user and the second ear assembly 12 over a second ear of the user such that the nozzle 16 can extend around a face of the user, from one ear to the other, and over at least the mouth of the user. Within each ear assembly 12, the rotation of the impeller 26 by the motor 32 will cause an airflow to be generated through the impeller casing 34 that draws air into the ear assembly 12 through the apertures in the outer cover 50. This flow of air will then pass through the filter assembly disposed between the outer cover 50 and the filter seat 46, thereby filtering and/or purifying the airflow. The resulting filtered airflow will then pass through the apertures 48 provided in the frustoconical portion of the filter seat 46 into the air passageway provided by the space between the impeller casing 34 and the opposing surface of the filter seat 46, with the air passageway then guiding the airflow to the air inlet 40 of the impeller casing 34. The impeller 26 will then force the filtered airflow out through the annular slot that provides the air outlet 42 of the impeller housing 36 and into the volute 38 of the impeller casing 34. The volute 38 then guides the filtered airflow through the air outlet 30 of the ear assembly 12 into the nozzle 16 through an air inlet 52, 54 provided by one of the open ends of the nozzle 16.

An issue with a head wearable air purifier as described above is that continuous breathing of a user onto the nozzle over a time period in which the air purifier is being used can increase the risk of bacterial growth on the nozzle. Various parts of the nozzle may also become contaminated with bacteria or other particles from the air over time. The nozzle therefore needs to be cleaned to remove such accumulated bacteria or other particles. Otherwise, there is a risk that use of the air purifier may not reduce, or may even increase, the level of harmful air pollutants that a user is exposed to. One way in which the filter assembly could be cleaned would be to wipe down its surfaces; however, this would be disadvantageous in that a cloth that is used for this may actually already include microbes that are then disposed onto the filter assembly, and also that it can be difficult to access the parts of a filter assembly to be cleaned in this way.

One way in which surfaces are decontaminated is by treating the surfaces with light. In particular, light of certain wavelengths is known to be very effective at killing any microbes that may have accumulated on the surfaces that are then illuminated by such light. Specifically, light in the far UVC portion of the electromagnetic spectrum is known to be effective for this purpose. The far UVC portion of the electromagnetic spectrum is typically defined as spanning the range of about 180-280 nm. The light used may therefore have a wavelength of about 222 nm, for instance. The use of far UVC light for this particular implementation brings a number of advantages that are not found in UV or near UV light. For example, the low energy electromagnetic light does not damage the material of the surfaces it illuminates. This is especially advantageous because many user appliances, devices or gadgets are at least partially made of plastics that are easily damaged by UV light. Another important advantage of far UVC light is that no direct line of sight between the light source and the surface or part to be cleaned is needed. Indirect irradiation of far UVC light helps to get rid of the microbial contamination too. Furthermore, this low energy electromagnetic light at 222 nm is generally safe for user interaction.

It is to be noted that emitting light in a far UVC portion of the electromagnetic spectrum as part of a decontamination process means that the emitted light contains a significant portion of light in that part of the electromagnetic spectrum and that the intensity of that significant portion is sufficient to have a useful anti-microbial and decontaminating effect. The emitted light does not need to be exclusively in the far UVC portion of the visual spectrum. As long as there is a sufficient intensity of light in that portion of the spectrum, and preferably at or around the 222 nm wavelength, for achieving a decontaminating effect, light from other parts of the electromagnetic spectrum may also be emitted. Further, it is noted that as part of the decontamination process the intensity of the emitted light may vary over time. Such variations may be gradual and continuous, or in the form of a pattern of light pulses. If pulsed light is used, the frequency, duration and intensity of the pulses may either be constant or varying.

It has been recognised that illuminating part or all of the surfaces of the nozzle of an air purifier with light in a far UVC portion of the electromagnetic spectrum can provide a decontamination or sterilisation effect. The nozzle is a part of the air purifier that particularly requires sterilisation because this is the part onto which a user most often breathes, coughs, sneezes, etc. It would be advantageous for a source of far UVC light to be provided in, or as part of, the air purifier so that decontamination of the nozzle may be performed when the air purifier is in use and being worn by a user. This would mean that continuous, intermittent or periodic decontamination could be performed without needing to wait until a user has finished using the air purifier (by which time a significant build-up of bacteria may have occurred, for instance). However, as will be apparent from FIGS. 1 and 2, it would be difficult, or impossible, to dispose a source of far UVC light on the nozzle of the air purifier in the vicinity of a part of the nozzle to be decontaminated (for instance, at the nozzle outlet) in a manner such that the part of the nozzle to be decontaminated would be illuminated directly by the light source for the purpose of decontamination.

The present invention is advantageous in that it provides a head wearable air purifier that overcomes these challenges to allow for decontamination of part or all of the nozzle of the air purifier while the air purifier is in use (or simply when it is being worn by the user). In particular, the present invention is advantageous in that light from a source of far UVC light—positioned in a part of the air purifier where space constraints are not so severe (namely, the ear assembly)—may be used to illuminate the nozzle for the decontamination thereof, as described in greater detail below.

In the broadest sense of the invention, there is provided a head wearable air purifier that has an ear assembly to be worn over a user's ear, and a nozzle attached to the ear assembly. The ear assembly has at least one light source for emitting light in a far UVC portion of the electromagnetic spectrum, where the light source is arranged to illuminate at least part of the nozzle for the decontamination thereof. That is, the constraints on space may be slightly less severe in certain parts of the ear assembly of the air purifier than in or on the nozzle. The light source is therefore disposed in the ear assembly in a way in which its emitted light can illuminate parts of the nozzle to be sterilised.

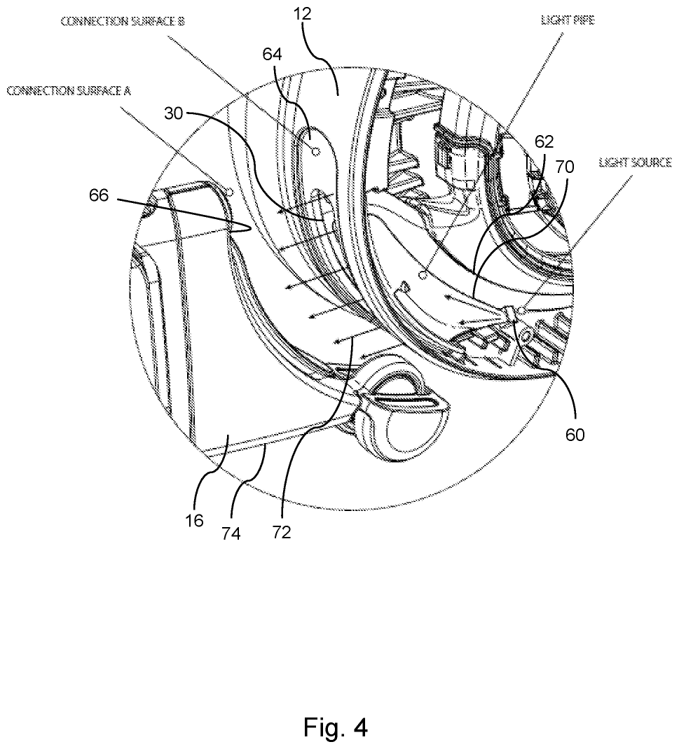

The light source may be disposed internally within the ear assembly 12 at a position in the vicinity of the air outlet 30 of the ear assembly 12. The light source may then be oriented to direct light emitted therefrom in a general direction of the air outlet 30, and therefore towards the nozzle 16. With reference to FIG. 4—which shows part of the view of FIG. 1 (specifically, the encircled part of FIG. 1)—in the described example the ear assembly 12 includes a far UVC light source in the form of a light emitting diode (LED) 60. The LED 60 is positioned near to the air outlet 30 and is oriented so that the emitted light shines towards the air outlet 30.

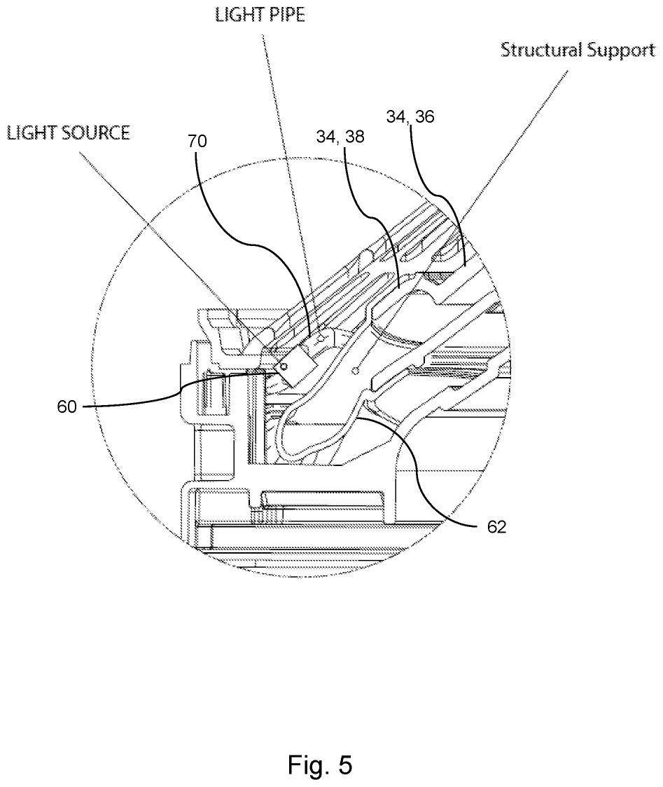

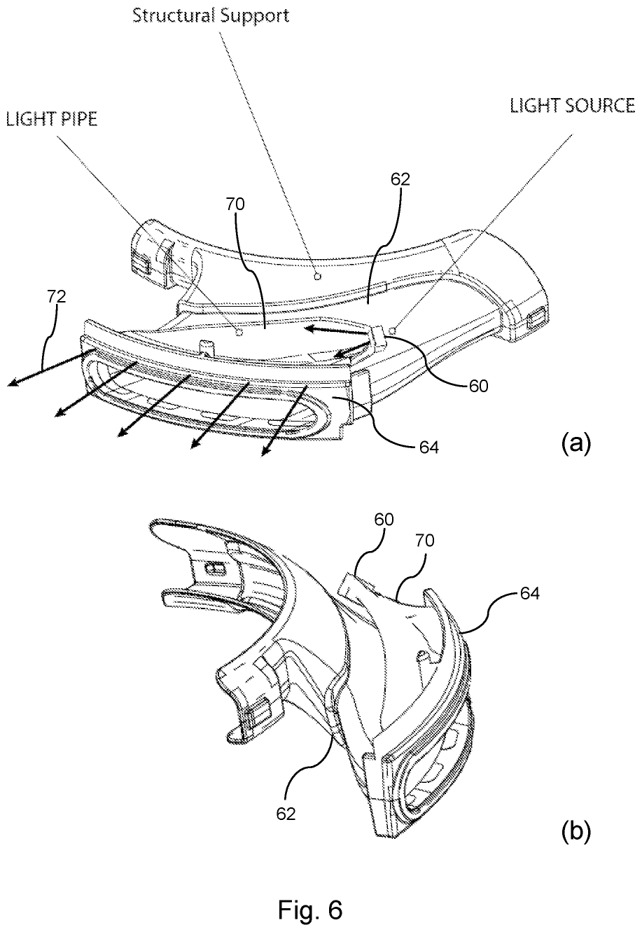

With additional reference to FIG. 5—which shows part of the view of FIG. 3 (specifically, the encircled part of FIG. 3)—in the described example the ear assembly 12 includes a structural support or attachment portion 62 that receives the filtered airflow from the volute 38 and provides the filtered airflow to the air outlet 30 of the ear assembly 12. With additional reference to FIGS. 6(a) and 6(b)—which show perspective views of the attachment portion 62—the attachment portion 62 includes a connection surface 64 that faces and abuts the nozzle 16 in order to allow the filtered airflow to be delivered to the nozzle 16. In particular, as is apparent from FIG. 4, the connection surface 64 of the ear assembly 12 abuts against a corresponding connection surface 66 of the nozzle 16 to ensure a properly sealed passage for the filtered airflow to pass from the ear assembly 12 into the nozzle 16.

In some examples of the present invention, it may be that a light source disposed in an ear assembly of the air purifier may not be able to directly irradiate a part of the nozzle that is to be sterilised with far UVC light. This may be because the light source cannot be oriented correctly to shine directly towards the nozzle, for packaging reasons for instance. Another issue with using a light source disposed in the ear assembly to sterilise the nozzle may be that the light emitted from the light source may shine in various different directions such that a sufficient amount of light for sterilisation does not reach the part of the nozzle to be sterilised. A further issue with such an arrangement is that there may be one or more (structural) components positioned between the light source and the part of the nozzle to be sterilised, meaning that the emitted light could be blocked from reaching its intended location.

To address one or more of these potential issues, the head wearable air purifier of the present invention may therefore be provided with a light guide arranged to guide light emitted from the at least one light source to illuminate the at least part of the nozzle to be decontaminated. As illustrated in the figures, in the described example the air purifier 10 includes a light guide 70 in the form of a light piping or a light lens. In particular, in the described example the light pipe is part of the ear assembly 12, and specifically is disposed adjacent to the attachment portion 62 of the ear assembly 12.

Such light pipes, e.g. clear tubes, are designed to carry light relatively short distances with high efficiency, and can be used to bend light around corners and in relatively tight spaces with minimal loss of light intensity. This therefore provides a simple and compact solution to guide the light to the desired location.

The light pipe 70 is arranged to receive light emitted by the LED 60 at one of its ends, and to guide the light towards the connection surface 64 of the ear assembly 12 where the filtered airflow passes from the ear assembly 12 to the nozzle 12. As is shown in FIGS. 6(a) and 6(b), the light pipe 70 may be narrower at its end nearest to, or adjacent to, the LED 60 than at its opposite end nearest to, or adjacent to, the connection surface 64. This may ensure that the light pipe 70 captures or receives most or all of the emitted light from the LED 70 and then guides the light to be spread across a width of the connection surface 64.

In some examples, the light piping 70 may extend through the connection surface 64 to allow the guided light to shine into or onto the nozzle 16. In the described example, however, the light from the LED 60 exits the light pipe 70 at a part of the attachment portion 62 opposite to (i.e. on the other side of) the connection surface 64. In order that the far UVC light may be provided to the nozzle 16, the part of the attachment portion 62 including the connection surface 64 is therefore formed from a material that allows the far UVC light to pass therethrough. For instance, the connection surface 64 may be formed from a (transparent) plastic material such as acrylic, polycarbonate or polypropylene, or may include the use of polylactic acid. The light exiting the light pipe 70 may then pass through the connection surface 64 generally in the direction indicated by the arrows 72 shown in FIGS. 4 and 6(a).

The light is therefore then received into the nozzle 16. As shown in FIGS. 1, 2 and 4, in the described example the nozzle 16 includes attachment portions 74, 76 defining its ends and being for attaching to the respective ear assemblies 12. The filtered air from the ear assemblies 12 is therefore received into the attachment portions 74, 76—via the air inlets 52, 54—of the nozzle 16. The nozzle 16 also includes a visor portion 78 that extends around a user's face in use, and includes the nozzle outlet 56 that delivers the filtered air to the user. The attachment portions 74, 76 are received into either end of the visor portion 78 and are attached to the visor portion 78. In some examples, there may be a gap or space defined between the outer surface of the attachment portions 74, 76 and the inner surface of the visor portion 78 when the attachment portions 74, 76 are received in the visor portion 78.

In the described example, the far UVC light received from the ear assembly 12 first passes through the connection surface 66 of the nozzle attachment portion. The connection surface 66 of the nozzle 16 may therefore also be formed from a material that allows the far UVC light to pass therethrough. The far UVC light received from the ear assembly 12 may additionally or alternatively pass through the gap defined between the nozzle attachment portions 74, 76 and the visor portion 78. The arrows 80 in FIG. 2 show a general direction and path followed by the far UVC light in the nozzle 16. Once the light has passed the nozzle attachment portions 74, 76 it is seen that the far UVC light may travel along, and illuminate, an internal surface of the nozzle 16 (in particular, the visor portion 78) to decontaminate this internal surface.

The sterilisation or decontamination process may be instigated in any appropriate manner. For instance, the head wearable air purifier 10 may include a switch (not shown) for switching the light source 60 between an on state, in which the light source 60 emits light, and an off state, in which the light source 60 does not emit light. The switch could for example be user operable. Alternatively, the light source 60 could be arranged to switch on when the motor-driven impeller is operational, which itself could be user operated. Further alternatively, the light source 60 could be arranged to switch on when the nozzle 16 is attached to the ear assemblies 12, or fixed to a particular (rotational) position relative to the ear assemblies 12, indicative that the air purifier 10 is in use. The light emitted from the light source 60 can be in any suitable manner, e.g. continuous, periodic, pulsed, etc.

Many modifications may be made to the examples described herein without departing from the scope of the appended claims.

In the above-described example, a single light source—in the form of a single LED—is provided in each of the ear assemblies. It will be understood that each ear assembly may include a number of light sources disposed at different locations within the ear assembly for providing sterilising far UVC light to the nozzle or to other parts of the air purifier. Also, although in the described example a single light pipe (having one end narrower than the other) is provided in each ear assembly, it will be understood that any number of light pipes, of any suitable shape, may be used to guide light emitted from the light source to the desired location, e.g. an inner surface of the nozzle.

In the above-described example, light piping is utilised in the ear assemblies of the air purifier. In different examples, light piping may additionally or alternatively be used in the nozzle. For instance, the light received from the ear assemblies may be received into light piping in the nozzle—e.g. disposed along an internal surface of the nozzle—and then guided to a location of the nozzle where sterilisation is desired, e.g. the air outlet of the nozzle.

In the above-described example, a light guide in the form of light piping is used to guide light from the light source in the ear assembly to a particular part of the nozzle. In different examples, different types of light guides may additionally or alternatively be used to guide the light in a desired manner. For instance, one or more suitably shaped/oriented reflective surfaces within the ear assemblies and or nozzle of the air purifier may be used to appropriately guide or direct the emitted light from the light source to the nozzle. As an example, an interior part of the nozzle may include suitably designed reflective surfaces to direct the light received from the ear assembly at the nozzle air input to the nozzle air output where sterilisation may be particularly desired.

In the above-described example, the filter assembly is housed in the ear assembly of the air purifier. In different examples, however, the filter assembly may be positioned elsewhere in or on the air purifier. For instance, the filter assembly may be located in or on the headband of the air purifier. Similarly, it will be understood that the motor and/or impeller could be disposed in a position other than in the ear assembly.

In some examples, one or both of the ear assemblies may include an acoustic driver unit (and associated components) such that the head wearable air purifier may additionally operate as traditional audio headphones.

1. A head wearable air purifier, comprising:
a filter assembly;
a motor-driven impeller for creating an airflow through the filter assembly to obtain a filtered airflow downstream of the filter assembly;
an ear assembly arranged to be worn over an ear of a user, the ear assembly comprising at least one light source for emitting light in a far UVC portion of the electromagnetic spectrum; and,
a nozzle attached to the ear assembly, the nozzle comprising a nozzle inlet arranged to receive the filtered airflow downstream of the filter assembly, and a nozzle outlet for emitting the received filtered airflow from the head wearable air purifier,
wherein the at least one light source is arranged to illuminate at least part of the nozzle for the decontamination thereof.

2. The head wearable air purifier according to claim 1, further comprising a light guide arranged to guide light emitted from the at least one light source to illuminate the at least part of the nozzle to be decontaminated.

3. The head wearable air purifier according to claim 2, wherein the light guide comprises at least one light pipe to guide light emitted from the at least one light source through the at least one light pipe.

4. The head wearable air purifier according to claim 2, wherein at least part of the light guide is disposed in the ear assembly.

5. The head wearable air purifier according to claim 2, wherein the light guide is arranged to guide the light emitted from the at least one light source to a connection surface of the ear assembly, the connection surface of the ear assembly being arranged to face a corresponding connection surface of the nozzle.

6. The head wearable air purifier according to claim 5, wherein the light guide is narrower at an end adjacent to the at least one light source than at an opposite end adjacent to the connection surface of the ear assembly.

7. The head wearable air purifier according to claim 5, wherein the connection surface of the ear assembly is formed from a material that allows the emitted light to pass therethrough.

8. The head wearable air purifier according to claim 5, wherein the connection surface of the ear assembly is formed from a plastics material, wherein the connection surface is formed from one or more of acrylic, polycarbonate, polypropylene or comprises the use of polylactic acid.

9. The head wearable air purifier according to claim 5, wherein the ear assembly comprises an attachment portion that comprises the connection surface and is for attaching the ear assembly to the nozzle, the attachment portion being arranged to receive the filtered airflow downstream of the filter assembly and to provide the received filtered airflow to the nozzle, wherein the at least one light source is disposed adjacent to the attachment portion.

10. The head wearable air purifier according to claim 9, wherein the at least one light source is attached to the attachment portion.

11. The head wearable air purifier according to claim 2, wherein at least part of the light guide is disposed in the nozzle.

12. The head wearable air purifier according to claim 2, wherein the light guide comprises one or more shaped features, for reflecting the emitted light, on an internal surface of the head wearable air purifier.

13. The head wearable air purifier according to claim 1, wherein the nozzle is arranged to receive light from the at least one light source in the ear assembly into an internal part of the nozzle.

14. The head wearable air purifier according to claim 13, wherein the nozzle comprises an attachment portion that comprises the nozzle inlet and is for attaching the nozzle to the ear assembly, and a visor portion comprising the nozzle outlet, and wherein the light from the at least one light source in the ear assembly is received into a space defined between the attachment and visor portions of the nozzle.

15. The head wearable air purifier according to claim 13, wherein light from the at least one light source in the ear assembly irradiates an internal surface of the nozzle.

16. The head wearable air purifier according to claim 1, wherein the head wearable air purifier comprises electrical switching arranged to switch between a closed state, in which the at least one light source is activated to emit light, and an open state, in which the at least one light source is deactivated and does not emit light.

17. The head wearable air purifier according to claim 16, wherein the electrical switching is arranged to automatically switch from the open state to the closed state upon the nozzle being attached to the ear assembly.

18. The head wearable air purifier according to claim 16, wherein in the closed state a motor of the air purifier is powered to drive the impeller to create the airflow through the filter assembly, and in the open state the motor does not drive the impeller.

19. The head wearable air purifier according to claim 16, wherein the nozzle is rotatable relative to the ear assembly when the nozzle is attached to the ear assembly, and wherein the electrical switching is arranged to automatically switch from the open state to the closed state upon the nozzle being moved from a first position to a second position, different from the first position, relative to the ear assembly.

20. The head wearable air purifier according to claim 1, wherein the nozzle is generally arcuate in shape.

21. The head wearable air purifier according to claim 1, wherein the filter assembly and the motor-driven impeller comprise part of the ear assembly, wherein the filter assembly and the motor-driven impeller are generally conical or frustoconical in shape.

22. The head wearable air purifier according to claim 1, wherein the at least one light source comprises one or more LEDs.

23. The head wearable air purifier according to claim 1, wherein the at least one light source is configured for emitting light having a wavelength of about 222 nm.

24. The head wearable air purifier according to claim 1, wherein:
the ear assembly comprises a first ear assembly arranged to be worn over a first ear of the user; and
the head wearable air purifier comprises a second ear assembly arranged to be worn over a second ear of a user, the second ear assembly comprising at least one second light source for emitting light in a far UVC portion of the electromagnetic spectrum, the at least one second light source being arranged to illuminate at least part of the nozzle for the decontamination thereof.

25. The head wearable air purifier according to claim 23, wherein the nozzle output is arranged, in use, adjacent to a user's mouth.
